(19) United States
(12) Patent Application Publication
(10) Pub. No.: US 2011/0097278 A1
(43) Pub. Date: Apr. 28, 2011

(54) HAIR STYLING COMPOSITIONS AND METHODS OF USE

(75) Inventor: Gilles M. Verboom, St. Charles, IL (US)

(73) Assignee: ALBERTO-CULVER COMPANY, Melrose Park, IL (US)

(21) Appl. No.: 12/746,726

(22) PCT Filed: Dec. 10, 2008
(86) PCT No.: PCT/US2008/086151

§ 371 Date: Jun. 7, 2010

Related U.S. Application Data
(60) Provisional application No. 61/013,916, filed on Dec. 14, 2007.

(51) Int. Cl. A61K 8/81; A61K 9/12; A61Q 5/06; B65D 47/00

(52) U.S. Cl. 424/43; 424/70.15; 222/544

(57) ABSTRACT

Provided is a hair-styling composition, which preferably includes a poly(vinylamine-vinylformamide) copolymer, a polyvinylpyrrolidone, and water, wherein the composition when applied to human hair, exhibits at least about 50% curl retention after about 2 hours under conditions of about 90% relative humidity and about 75° F. Also provided are methods for styling mammalian hair and controlling frizz with the composition of the present invention, and a personal care product that includes the composition of the present invention.

### BACKGROUND OF THE INVENTION

Hair styling or hair setting compositions are widely used by consumers in the cosmetic industry to retain a particular shape or style of the hair. Hair styling compositions can assist in manipulating or styling the hair, providing temporary benefits in holding the shape of the hairstyle (fixing) and/or maintaining the shine or appearance (grooming, restyling) of the hair, e.g., in the evening, during the day, between hair washing periods, or between subsequent hair setting procedures.

Various objective and subjective methods are used to measure the efficacy of a hair-styling composition. One method commonly employed to test the efficacy of hair styling compositions involves measuring curl retention under humid conditions. In this methodology, hair tresses are curled either before or after applying the hair styling product to the hair, and the ability of the product to retain curl (curl retention) can be monitored periodically during exposure to a controlled temperature and relative humidity (RH). For instance, the retention of hair rolled into curls of ¾ inch diameter prepared by applying 0.3 ml of a styling gel onto 1.8 gm hair tresses can be measured under controlled temperatures, e.g., in the range of about 70° F. to about 85° F., and high relative humidity, e.g., in about 90% RH. Testing under such conditions can be used to measure properties sometimes referred to as high humidity curl retention (or HHCR).

Hair styling compositions also can be subjectively evaluated, e.g., by visual and tactile sensory methods (e.g., by visual examination and touching) for characteristics such as appearance (shine, cleanliness, naturalness of appearance and texture), feel (stiffness, tackiness, softness), curl memory (bounce, and restylability), ease of combing and brushing the hair, residue (flaking), static, smoothness, and the like. Also of importance are the aesthetic characteristics and appearance provided by hair styling compositions before, during, and after application to hair. Preferably, the product viscosity should be non-runny to avoid dripping during application. The product should be easy to spread, have a smooth texture, a non-tacky feel, and be able to dry relatively quickly on the hair.

Of further importance is the ability of hair styling compositions to control hair “frizz,” which generally causes hair to become unmanageable and appear undisciplined. Frizz can become a problem when hair is exposed to higher humidity, e.g., a relative humidity of about 80% or more. The problem can worsen in people with curly hair, either naturally or “permed,” leading to what many have termed a “bad hair day.” In such a case, hair loses its natural shape and/or its curl definition. Hair is often subjected to a wide variety of stresses that can cause damage to the hair, resulting in frizz. These include shampooing, rinsing, drying, heating, combing, styling, perming, coloring, exposure to the elements, and the like. Such stresses can leave the hair in a dry, rough, lusterless, or frizzy condition, which can be caused, e.g., by repeated abrasion of the hair surface and removal of the hair's natural oils and other natural conditioning and moisturizing components. Additionally, hair is often subjected to weather-related stresses, e.g., sunlight, wind, and changes in temperature and humidity, which can cause hair frizz and other conditions considered by consumers to be cosmetically undesirable.

Hair-setting compositions that include one or more hair-setting polymers to impart styling and/or fixative properties have been disclosed. For example, U.S. Pat. No. 4,713,236 describes compositions that include amine-containing polymers and copolymers that contain a primary pendant amine group, for imparting conditioning properties to hair. U.S. Pat. Nos. 5,478,553 and 5,632,977 describe hair fixative compositions containing polymeric n-vinyl formamide and methods of treating hair. U.S. Published Patent Application No. 2007/0110690 describes a process for inhibiting hair from becoming frizzy that involves contacting hair with anionic silicone and with polyvinylamine.

Providing hair styling compositions that exhibit good high humidity curl retention while maintaining desirable subjective properties, e.g., smooth texture, curl memory, bounce, naturalness of appearance, etc., has been difficult to achieve with conventional hair-setting compositions. There is an ongoing need for hair styling compositions that provide high humidity curl retention and resistance to frizziness, as well as desirable subjective properties. The present invention provides such compositions and associated methods of use.

### BRIEF SUMMARY OF THE INVENTION

The present invention provides a hair-styling composition that, when applied to mammalian hair, imparts at least about 50% curl retention in the hair after about 2 hours under conditions of about 90% relative humidity and a temperature about 75° F., and yet leaves the hair with desirable subjective properties. The composition of the present invention preferably includes one or more poly(vinylamine-vinylformamide) copolymers as a styling polymer, and more preferably a includes a linear poly(vinylamine-vinylformamide) copolymer, a polyvinylpyrrolidone and an aqueous carrier.

The present invention also provides a method for styling mammalian hair, which method includes applying to the hair a styling-effective amount of the composition of the present invention. The present invention additionally provides a method for controlling frizz in mammalian hair, which method includes applying to the hair with a fizz-controlling effective amount of the composition of the present invention.

The invention further provides a personal care product that includes a container, a valve associated with the container, and the composition of the present invention contained within the container, wherein the valve is adapted to dispense the composition therefrom.

### BRIEF DESCRIPTION OF THE DRAWINGS

FIG. 1 depicts curl retention of exemplary compositions over time at high relative humidity.

FIG. 2 depicts curl retention of exemplary compositions over time at high relative humidity.

FIG. 3 depicts curl retention of exemplary compositions over time at high relative humidity.

FIG. 4 depicts curl retention of exemplary compositions over time at high relative humidity.

### DETAILED DESCRIPTION OF THE INVENTION

When applied to mammalian (e.g., human) hair, the composition of the present invention exhibits at least about 50% curl retention after about 2 hours under conditions of about 90% relative humidity and a temperature of about 75° F. The composition of the present invention also exhibits good fizz control under conditions of high relative humidity.

The composition of the present invention preferably includes one or more poly(vinylamine-vinylformamide) copolymers as a styling polymer, and more preferably includes one or more linear poly(vinylamine-vinylformamide) copolymers, a polyvinylpyrrolidone polymer and an aqueous carrier. The poly(vinylamine-vinylformamide) copolymer is preferably present in the composition in a hair-styling effective amount, e.g., in an amount effective to promote at least about 50% curl retention in the hair after about 2 hours under conditions of about 90% relative humidity and a temperature about 75° F., when the composition is applied to mammalian hair. Suitable poly(vinylamine-vinylformamide) copolymers can be obtained, e.g., by partial hydrolysis of a polyvinylformamide, to produce one or more copolymers that contain vinylamine and vinylformamide monomeric units. Poly(vinylamine-vinylformamide) copolymers, which can be used as a styling polymer in the composition of the present invention, include the polymers contained in products sold under the trademark LUPAMIN®, which are sold by BASF and are supplied as aqueous solutions containing linear poly(vinylamine-vinylformamide) copolymers. The polymers in LUPAMIN® are prepared by polymerization of vinylformamide followed by partial hydrolysis of the polyvinylformamide. Exemplary poly(vinylamine-vinylformamide) copolymers, which can be used in the composition of the present invention, include the polymers contained in LUPAMIN® 9095, LUPAMIN® 9050, LUPAMIN® 9030, LUPAMIN® 9010, LUPAMIN® 5095 and LUPAMIN® 1595.

The digits used in conjunction with the LUPAMIN® product name correspond to the molecular weight and the extent of hydrolysis of the polymer. The first two (i.e., first and second) digits in the product name correspond to the polymer molecular weight. For instance, the first two digits in LUPAMIN® 9095, LUPAMIN® 9050, LUPAMIN® 9030 and LUPAMIN® 9010, i.e., “90,” are indicative of the polymer molecular weight polymer. The average molecular weights of exemplary polymers and other properties associated with corresponding LUPAMIN® products, as published in BASF's technical bulletins, are summarized below in Table A.

TABLE A

| | Lupamin ® 9095 | Lupamin ® 9030 | Lupamin ® 9010 | Lupamin ® 5095 | Lupamin ® 1595 |
|---|---|---|---|---|---|
| Form | Liquid | Liquid | Liquid | Clear Pale | Clear Pale |
| | | | | Yellow Liquid | Yellow Liquid |
| Density (g/mL) | 1.08 | 1.08 | 1.08 | 1.08 | 1.08 |
| % Solids (wt %) | 20-22% | 16-18% | 13-15% | 21-24% | 28-32% |
| % Polymer (wt %) | 6-8 | 10-12 | 12-14* | 8-12 | 9-11 |
| Ave. Molecular | 340,000 | 340,000 | 340,000 | 45,000 | <10,000 |
| Weight (g/mol) | | | | | |
| Viscosity (mPas | >5000 | <5000 | <5000 | <1000 | <1000 |
| at 20° C.) | | | | | |
| pH | 7-9 | 7-9 | 7-9 | 7-9 | 7-9 |

*estimated based on solids content, hydrolysis index and polymer content relative to total solids reported for other LUPAMIN ® products

LUPAMIN® 9050 is believed to have a molecular weight of 340,000 based on the molecular weights reported in BASF's technical bulletins for LUPAMIN® 9095, 9030 and 9010. LUPAMIN® 9050 is believed to have a solids content of about 16-19 wt % based on the results of solids testing performed on a product sample and solids content reported for LUPAMIN® 9095, 9030 and 9010. LUPAMIN® 9050 is estimated to have a poly(vinylamine-vinylformamide) copolymer content (i.e., polymer content) of about 9-12 wt % based on solids testing, and reported solids and polymer content for other LUPAMIN® products.

The last two (i.e., third and fourth) digits used in conjunction with the LUPAMIN® product name represent the “hydrolysis index,” which corresponds to the percent of the formamide functional groups in the polymer that have been hydrolyzed and converted into vinylamine units. For instance, the last two digits in LUPAMIN® 9095, i.e., “95,” indicate the degree of hydrolysis, i.e., that the polymer is about 95% hydrolyzed (or greater than 90% hydrolyzed as noted in BASF's technical bulletins for Lupamin® 5095 and Lupamin® 1595). Thus, the polymers contained in LUPAMIN® 9095, LUPAMIN® 5095 and LUPAMIN® 1595 are believed to contain about 95% vinylamine monomeric units (vinylamine monomers) and about 5% vinylformamide monomeric units (vinylformamide monomers). By contrast, LUPAMIN® 9050 is believed to contain about 50% vinylamine monomers and about 50% vinylformamide monomers, LUPAMIN® 9030 is believed to contain about 30% vinylamine monomers and about 70% vinylformamide monomers, and LUPAMIN® 9010 is believed to contain about 10% vinylamine monomers and about 90% vinylformamide monomers.

The composition of the present invention also can include two or more poly(vinylamine-vinylformamide) copolymers. In some embodiments, combinations of two or more poly(vinylamine-vinylformamide) copolymers have been found to promote unexpectedly superior high humidity curl retention properties. In one embodiment, the composition of the present invention includes at least one high molecular weight poly(vinylamine-vinylformamide) copolymer and at least one low molecular weight poly(vinylamine-vinylformamide) copolymer. As used herein, a high molecular weight poly(vinylamine-vinylformamide) copolymer refers to a poly(vinylamine-vinylformamide) copolymer with an average molecular weight greater than about 100,000 g/mole and a low molecular weight poly(vinylamine-vinylformamide) copolymer refers to a poly(vinylamine-vinylformamide) copolymer with an average molecular weight of about 100,000 g/mole or less.

The high molecular weight poly(vinylamine-vinylformamide) copolymer can include, e.g., at most about 95% vinylamine monomers (e.g., about 95% vinylamine monomers and about 5% vinylformamide monomers), at most about 50% vinylamine monomers (e.g., about 50% vinylamine monomers and about 50% vinylformamide monomers), at most about 30% vinylamine monomers (e.g., about 30% vinylamine monomers and about 70% vinylformamide monomers), or at most about 10% vinylamine monomers (e.g., about 10% vinylamine monomers and about 90% vinylformamide monomers). Suitable high molecular weight poly(vinylamine-vinylformamide) copolymers include, for example, LUPAMIN® 9095, LUPAMIN® 9050, LUPAMIN® 9030 and LUPAMIN® 9010 polymers. The low molecular weight poly(vinylamine-vinylformamide) copolymer can include, e.g., at most about 95% vinylamine monomers (e.g., about 95% vinylamine monomers and about 5% vinylformamide monomers). Suitable low molecular weight poly(vinylamine-vinylformamide) copolymers include, e.g., LUPAMIN® 5095 and LUPAMIN™ 1595 polymers.

The poly(vinylamine-vinylformamide) copolymer can be present in the composition of the present invention, e.g., in an amount of from about 0.01 wt % to about 10 wt %, from about 0.05 wt % to about 10 wt %, from about 0.1 wt % to about 10 wt %, from about 0.1 wt % to about 10 wt %, from about 0.1 wt % to about 5 wt %, or from about 0.1 wt % to about 2 wt %.

The composition of the present invention preferably also includes polyvinylpyrrolidone (PVP), which has been found to provide the hair with good styling performance and desirable subjective properties such as, e.g., gloss, low flaking and smooth texture, without sacrificing high humidity curl retention or resistance to frizz. The composition of the present invention can include polyvinylpyrrolidone, e.g., in an amount of from about 0.01 wt % to about 20 wt %, from about 0.05 wt % to about 15 wt % polyvinylpyrrolidone, from about 0.1 wt % to about 10 wt % polyvinylpyrrolidone, from about 0.1 wt % to about 5 wt % polyvinylpyrrolidone, from about 0.1 wt % to about 1 wt % polyvinylpyrrolidone, or from about 0.5 wt % to about 1 wt % polyvinylpyrrolidone.

The composition of the present invention can further include one or more additional ingredients such as, for example, a conditioning agent, a film former or modifier (in addition to PVP), a thickener, a surfactant, an emollient, an emulsifier, a propellant, and the like, and combinations thereof. The composition of the present invention preferably exists in the form of a mousse or a gel.

Suitable additional film formers can include, e.g., vinylpyrrolidone copolymers, cationic cellulose derivatives, polyurethanes, acrylates/hydroxyester acrylate copolymer, celluloses and polysaccharide gums and their derivatives and the like, and combinations thereof. The composition of the present invention can include, for example, from about 0.01 wt % to about 10 wt % of one or more additional film formers, from about 0.05 wt % to about 5 wt % of one or more additional film formers, or from about 0.1 wt % to about 5 wt % of one or more additional film formers. Suitable film forming polymers also can include, e.g., one or more nonionic copolymers of N-vinylpyrrolidone, methacrylamide and N-vinylimidazole.

Suitable film forming polymers also can include, e.g., one or more copolymers of vinylpyrrolidone and dimethylaminoethyl methacrylate(s). The composition of the present invention can include, e.g., from about 0.01 wt % to about 15 wt % of one or more film forming vinylpyrrolidone copolymers, e.g., from about 0.05 wt % to about 10 wt % of one or more film forming vinylpyrrolidone copolymers, or from about 0.1 wt % to about 10 wt % of one or more film forming vinylpyrrolidone copolymers. Exemplary film forming vinylpyrrolidone copolymers include LUVISET® CLEAR, available from BASF, and VP/dimethylaminoethyl methacrylate copolymer 845-G.

Suitable film formers further can include, e.g., cationic cellulose derivatives. The composition of the present invention can include, for example, from about 0.01 wt % to about 10 wt % of one or more cationic cellulose derivatives, from about 0.02 wt % to about 5 wt % of one or more cationic cellulose derivatives, or from about 0.05 wt % to about 5 wt % of one or more cationic cellulose derivatives. A preferred class of cationic cellulose derivatives include copolymers of a hydroxyethylcellulose and diallyldimethyl ammonium chloride. An exemplary cationic cellulose derivative is polyquaternium-4, a copolymer of cellulose, 2-hydroxyethyl ether and diallyldimethyl ammonium chloride. Polyquaternium-4 is the active ingredient in products marketed under the names CELQUAT® H-100 and CELQUAT® L-200. It will be appreciated that some film formers, e.g., CELQUAT® H-100 also may function as conditioning agents.

Suitable film modifiers can include, for example, one or more aminosilicones, one or more PEG-n dimethicones, one or more PEG-n/PPG-n dimethicones, one or more cyclomethicones, one or more plasticizers (e.g., glycols, glycol ethers, glycerine), and the like, and combinations thereof. Suitable dimethicones can include polyethylene/propylene glycol derivatives of dimethicone containing an average of n moles of ethylene/propylene oxide, e.g., where n is in the range of about 3 to about 20. An exemplary PEG-n/PPG-n dimethicone includes a PEG-18/PPG-18 dimethicone, available from Dow Corning under the trade name DC-190. The composition of the present invention can include, e.g., from about 0.01 wt % to about 10 wt % of one or more film modifiers, from about 0.02 wt % to about 5 wt % of one or more film modifiers, or from about 0.05 wt % to about 5 wt % of one or more film modifiers.

Suitable thickeners can include, e.g., one or more associative and non-associative thickeners, one or more polysaccharides, polysaccharide derivatives, gums (e.g., guar gum, xanthan gum), and the like, and combinations thereof. Suitable associative thickeners can include, e.g., acrylates/beheneth-25 acrylate copolymers, polyether-1/1,3-butylene glycol blends, and combinations thereof. The composition of the present invention can include, for example, from about 0.01 wt % to about 15 wt % of one or more thickeners, from about 0.05 wt % to about 8.0 wt % of one or more thickeners, or from about 0.1 wt % to about 3.0 wt % of one or more thickeners. Exemplary thickeners include TINOVIS® GTC, available from Ciba Specialty Chemicals, PURE THIX® HH, available from Southern Clay, and combinations thereof.

The aqueous carrier can include any suitable quantity of water, e.g., from about 25 wt % to about 97 wt % water (e.g., from about 30% to about 95% water). Preferably, the composition of the present invention includes from about 50 wt % to about 97 wt %, and more preferably from about 70 wt % to about 90 wt %, and most preferably from about 80 wt % to about 90 wt % water. Preferably, the water used in the composition of the present invention is deionized water.

Suitable conditioning agents can include, for example, one or more amphoteric copolymers, one or more amphoteric terpolymers, one or more cationic conditioners and the like, and combinations thereof. Suitable conditioning agents can include amphoteric terpolymers of acrylic acid, diallyl dimethyl ammonium chloride, and acrylamide. The composition of the present invention can include, for example, from about 0.01 wt % to about 20 wt % of one or more conditioning agents, from about 0.01 wt % to about 15 wt % of one or more conditioning agents, or from about 0.05 wt % to about 10 wt % of one or more conditioning agents. An exemplary conditioning agent is polyquaternium-39, sold under the tradename MERQUAT® PLUS 3330, available from Nalco Co. Other exemplary products that may serve as conditioning agents include polyquaternium-4 and/or VP/dimethylaminoethyl methacrylate copolymer 845-G.

Suitable surfactants can include, e.g., one or more anionic, nonionic, cationic, and amphoteric surfactants, with nonionic, cationic, and amphoteric surfactants being preferred. Exemplary surfactants include PPG-5/Ceteth 20, Oleth-20, polysorbate-20, and cocamidopropyl betaine. The composition of the present invention can include, for example, from about 0.01 wt % to about 20 wt % of one or more surfactants, from about 0.01 wt % to about 15 wt % of one or more surfactants, or from about 0.05 wt % to about 10 wt % of one or more surfactants.

The hair styling composition of the present invention can include other components that may be suitable for use in conventional hair styling compositions such as, e.g., conventional hair fixative, hair setting and/or hair grooming gels, rinses, emulsions (oil-in-water, water-in-oil or multiphase), lotions, creams, pomades, sprays (pressurized or non-pressurized), spritzes, mousses, foams, shampoos, and solids (e.g., as sticks, semisolids and the like).

If desired, the composition of the present invention can include a propellant, e.g., for dispensing the composition (e.g., in the form of a mousse or gel). The composition of the present invention can include, for example, from about 0.01 wt % to about 20 wt % of one or more propellants, from about 0.01 wt % to about 15 wt % of one or more propellants, or from about 0.05 wt % to about 10 wt % of one or more propellants. Exemplary propellants include propane, butane, and mixtures thereof.

In some embodiments, the hair-styling composition of the present invention exhibits at least about 60% curl retention after about 2 hours under conditions of about 90% relative humidity and a temperature of about 75° F. In other embodiments, the hair-styling composition of the present invention exhibits at least about 60% curl retention after about 10 hours under conditions of about 90% relative humidity and a temperature of about 75° F. In yet other embodiments, the hair-styling composition of the present invention exhibits at least about 80% curl retention after about 2 hours under conditions of about 90% relative humidity and a temperature of about 75° F. In still yet other embodiments, the hair-styling composition of the present invention exhibits at least about 80% curl retention after about 10 hours under conditions of about 90% relative humidity and a temperature of about 75° F.

The present invention further provides a method of styling mammalian hair, which preferably includes applying to the hair a styling-effective amount of the composition of the present invention and styling the hair. The composition can be applied in any suitable manner, e.g., by working the composition through the hair, e.g., with the hands and fingers or with a suitable implement such as, e.g., a comb or brush, to ensure uniform coverage.

The present invention further provides a method of controlling fizz, which preferably includes applying to mammalian hair a frizz-controlling effective amount of the composition of the present invention. Consumers often associate fizz, e.g., the tendency of individual hairs to stray from alignment or conformity with one another, with an unruly or undesirable appearance. The styling composition of the present invention can be use to effectively control (e.g., inhibit, reduce or ameliorate) frizz in mammalian hair.

The present invention further provides a personal care product comprising a container, a valve associated with the container, and the composition of the present invention contained within the container, wherein the valve is suitably adapted to dispense the composition from the container. The product may include instructions for applying the composition to style the hair and/or control frizz.

The following examples further illustrate the invention but, of course, should not be construed as in any way limiting its scope.

### Example 1

This example illustrates a process for preparing exemplary gel compositions.

The gels are prepared by mixing three separate blends. The ingredients and amounts used (in wt % relative to the total weight of the composition) are summarized in Table 1.

The blends are named phase A, B and C. T hey are prepared at room temperature as follows. The batches of gel prepared were 500 gms in size.

Phase A is prepared in a 500 ml beaker equipped with a mixer. First the De-Ionized (DI) water is added, the mixer is turned on to create a vortex, then Celquat H-100 from National Starch is slowly added in such a way as to fall onto the wall of vortex. When the addition is completed, the mixer speed is reduced to remove the vortex but mixing is kept until the Celquat H-100 is fully dissolved. Mixing may take up to 2 hours. Then Tinovis GTC is added and while mixing at the same slow rate. Gentle mixing is maintained.

Phase B is prepared also in a 500 ml beaker equipped with a mixer. DI water is added first under gentle mixing and each ingredient per Table 1 are added successively in the order provided. The amounts of LUPAMIN® shown in Table 1 represent the amount of solution as supplied by BASF. Gentle mixing is maintained.

Phase C is prepared in a small 20 ml beaker. The Oleth-20 is pre-warmed to a liquid/pasty state and mixed in with a small spatula with PPG-5-Ceteth-20 the fragrance and Dow Corning DC-190 and Croda Procetyl AWS. Mix by hand until clear.

When Phase C is completed, it is added to Phase A while mixing. When homogeneous, the blend of Phase B and C was added to Phase A. It was mixed gently until homogeneous and the final ingredient Angus (Dow Corning) AMP-95 was then added. Gentle mixing was kept for ten minutes. The pH was measured and recorded. The viscosity was measured the next day with a Brookfield viscosimeter on samples left in a water bath overnight thermostated at 25C

TABLE 1

| Hair Styling | | Gel 1.A | | Gel 1.B |
|---|---|---|---|---|
| Composition | Phase | (wt %) | Phase | (wt %) |
| Deionized water | A | 40.32 | A | 40.32 |
| Cationic cellulose | A | 0.50 | A | 0.50 |
| derivative (Poly- | | | | |
| quaternium-4) | | | | |
| Acrylates/beheneth-25 | A | 3.00 | A | 3.00 |
| acrylate copolymer | | | | |
| (Tinovis ® GTC) | | | | |
| DMDM hydantoin | A | 0.15 | A | 0.15 |
| Deionized water | B | q.s. to 100 | B | q.s. to 100 |
| Polyether-1/1,3 butyl- | B | 0.25 | B | 0.25 |
| ene glycol (Pure | | | | |
| Thix ® HH) | | | | |
| PVP (20% solution) | B | 4.00 | B | 4.00 |
| Vinylpyrrolidone | B | 3.50 | B | 3.50 |
| copolymer (20 wt %) | | | | |
| Luviset ® Clear | | | | |
| TRIISOPROPANOL- | B | 0.70 | B | 0.70 |
| AMINE (85 wt %) | | | | |
| DMDM hydantoin | B | 0.15 | B | 0.15 |
| TETRASODIUM | B | 0.02 | B | 0.02 |
| EDTA | | | | |
| LUPAMIN ® 1595 | B | 0.50 | B | |
| LUPAMIN ® 9095 | B | | B | 0.50 |
| Propylene glycol | B | 1.00 | B | 1.00 |
| Sorbitol (70%) | B | 0.50 | B | 0.50 |
| Violet Dye (prediluted | C | 0.40 | C | 0.40 |
| to 0.01%) | | | | |
| Surfactant (PPG-5- | C | 0.10 | C | 0.10 |
| Ceteth-20) | | | | |
| Surfactant (Oleth-20) | C | 0.40 | C | 0.40 |
| Fragrance | C | 0.10 | C | 0.10 |
| Film Modifier (PEG- | C | 0.10 | C | 0.10 |
| 18/PPG-18 Dimethi- | | | | |
| cone) | | | | |
| Aminomethyl- | | 0.10 | | 0.10 |
| propanol (95 wt %) | | | | |
| | | pH 7.6 | | pH 7.5 |

| | | |
|---|---|---|
| Helipath RV T-C 5 rpm 1 mn | 630000 | 101000 |
| downward | | |

### Example 2

This example illustrates a process for preparing an exemplary mousse composition.

The ingredients and amounts used (in wt % relative to the total weight of the composition) are summarized in Table 2. The amount of LUPAMIN® 9095 represents the amount of solution as supplied by BASF. Batches of 500 gms each in a 750 ml beaker were prepared at room temperature as follows. First the DI water was added and the mixer turned on at low speed. The first two ingredients were added and mixed until the solution is cleared. The mixer speed is increased to create a vortex. The cationic cellulose powder was added slowly and carefully to fall onto the wall of the vortex; when the addition is completed, the mixer speed was reduced to remove the vortex. When the solution becomes clear, the next ingredients were added successively one at a time, each addition taking place when the solution became homogeneous looking. The last two ingredients were premixed until clear and added at the end of the blending process. Mixing was maintained for 15 minutes after the addition of the premix. The material was transferred into a jar equipped with a cap and left overnight into a thermostated water bath maintained at 25C. pH and viscosity (Brookfield Helipath RV T-C 5 rpm 1 nm downward) were measured the next day.

TABLE 2

| | Addition | Gel 2 |
|---|---|---|
| Hair Styling Composition | Order | (wt %) |
| Deionized water | 1 | q.s. to 100 |
| Sorbitol | 2 | 0.010 |
| PVP | 3 | 0.669 |
| Cationic cellulose derivative | 4 | 0.25 |
| (Polyquaternium-4) | | |
| Vinylpyrrolidone | 5 | 1.798 |
| copolymer (20 wt%) | | |
| Luviset ® Clear | | |
| LUPAMIN ® 9095 | 6 | 5.475 |
| OLEALKONIUM CHLORIDE | 7 | 0.368 |
| GLYCERINE, CP/USP | 8 | 0.011 |
| HYDROLYZED ELASTIN | 9 | 0.011 |
| COCAMIDOPROPYL BETAINE, | 10 | 0.022 |
| WATER, SODIUM CHLORIDE | | |
| GELATIN/KERATIN AMINO | 11 | 0.011 |
| ACIDS/LYSINE HYDROXYPRO- | | |
| PYL TRIMONIUM CHLORIDE | | |
| PPG-12-PEG-50 LANOLIN | 12 | 0.198 |
| PROPYLENE GLYCOL, USP | 13 | 0.002 |
| CYCLOMETHICONE | 14 | 0.030 |
| POLYSORBATE 20, WATER | 15 | 0.763 |
| FRAGRANCE | 16 | 0.382 |
| Next day pH = 7.95 Vis = 48000 | | |

### Example 3

This example demonstrates curl retention under high humidity conditions of exemplary hair styling compositions. In this methodology, hair tresses were curled either after applying the hair styling product to the hair. Curls of ¾ inch diameter were prepared by applying 0.3 ml of gel onto 1.8 gm hair tresses and dried overnight in an oven at 54C. The next day, the tresses were left to equilibrate at room temperature for one hour, then they were stored in an environmental room with controlled temperature and relative humidity (RH). The ability of the composition to retain curl (curl retention) was monitored periodically by measuring the length of the tresses. Typical conditions used were 90% RH at 75F temperature. It is sometimes referred to as high humidity curl retention (HHCR).

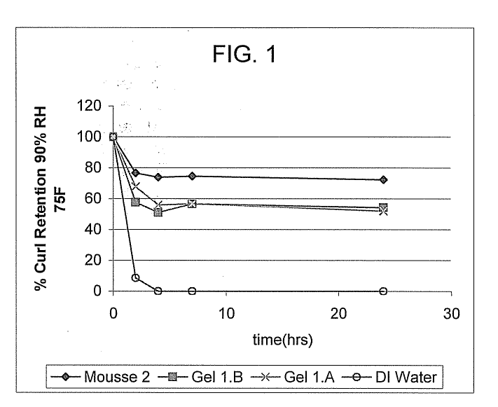

FIG. 1 describes the test results for percent curl retention over time for Gels 1.A, 1.B and 2. All example compositions achieved at least about 60% curl retention after about 2 hours under conditions of about 90% relative humidity at about 75° F.

### Example 4

This example illustrates a process for preparing exemplary mousse compositions.

The ingredients and amounts used (in wt % relative to the total weight of the final composition) are summarized in Table 3. The amounts of LUPAMIN® represent the amount of solution as supplied by BASF. Batches of 500 g of concentrate (each in a 750 ml beaker were prepared at room temperature as follows. First the DI water was added and the mixer turned on at low speed. The first two ingredients were added and mixed until the solution is cleared. The mixer speed is increased to create a vortex. The cationic cellulose powder was added slowly and carefully to fall onto the wall of the vortex; when the addition is completed, the mixer speed was reduced to remove the vortex. When the solution becomes clear, the next ingredients were added successively one at a time, each addition taking place when the solution became homogeneous looking. The last two ingredients were premixed until clear and added at the end of the blending process. Mixing was maintained for 15 minutes after the addition of the premix. The material was transferred to a jar equipped with a cap and left overnight into a thermostated water bath maintained at 25C. The pH and viscosity (Brookfield LV, Spindle #1, 60 or 12 rpm, 1 mn) were measured the next day.

The mousse cans were prepared using aluminum 53/195 mm standard cans equipped with standard mousse valve and by adding 188.4 g of concentrate first to the can, crimping the valve and then adding 11.6 g of propellant A-60. This corresponds to 5.8% propellant in the finished product.

TABLE 3

| | Mousse 4.1 | Mousse 4.2 | Mousse 4.3 |
|---|---|---|---|
| Hair Styling Composition | (wt %) | (wt %) | (wt %) |
| Deionized water | 85.621 | 86.713 | 85.621 |
| Sorbitol | 0.010 | 0.010 | 0.010 |
| PVP | 0.595 | 0.595 | 0.595 |
| Cationic cellulose derivative | | 0.506 | |
| (Polyquaternium-4) | | | |
| Vinylpyrrolidone | 1.598 | | 1.598 |
| copolymer (20 wt %) | | | |
| Luviset ® Clear | | | |
| LUPAMIN ® 1595 | 1.324 | 1.324 | 1.324 |
| LUPAMIN ® 9095 | 3.782 | | |
| LUPAMIN ® 9050 | | 3.782 | 3.782 |
| GLYCERINE, CP/USP | 0.010 | 0.010 | 0.010 |
| HYDROLYZED ELASTIN | 0.010 | 0.010 | 0.010 |
| COCAMIDOPROPYL | 0.020 | 0.020 | 0.020 |
| BETAINE, WATER, | | | |
| SODIUM CHLORIDE | | | |
| GELATIN/KERATIN | 0.010 | 0.010 | 0.010 |
| AMINO ACIDS/LYSINE | | | |
| HYDROXYPROPYL | | | |
| TRIMONIUM CHLORIDE | | | |
| PPG-12-PEG-50 LANOLIN | 0.176 | 0.176 | 0.176 |
| PROPYLENE GLYCOL, | 0.002 | 0.002 | 0.002 |
| USP | | | |
| CYCLOMETHICONE | 0.027 | 0.027 | 0.027 |
| POLYSORBATE 20, | 0.678 | 0.678 | 0.678 |
| WATER | | | |
| FRAGRANCE | 0.339 | 0.339 | 0.339 |
| ISOBUTANE (AND) | 5.800 | 5.800 | 5.800 |
| PROPANE (Propellant) | | | |

### Example 5

This example demonstrates high humidity curl retention properties of exemplary compositions. A methodology as described for the Gels in Example 3 was employed, except that 0.3 g of mousse is applied on the 1.8 g hair tresses.

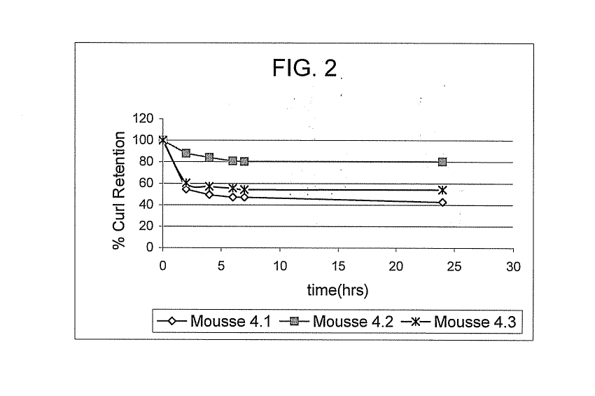

FIG. 2 describes the test results for percent curl retention over time exhibited by Mousse 4.1, Mousse 4.2 and Mousse 4.3, all of which achieved at least about 60% curl retention after about 2 hours under conditions of about 90% relative humidity at about 75° F.

### Example 6

The example is another illustration of the compositions described in Table 4 prepared using a process as described in Example 4.

The ingredients and amounts used (in wt % relative to the total weight of the composition) are summarized in Table 4. The amounts of LUPAMIN® represent the amount of solution as supplied by BASF.

TABLE 4

| | Mousse | Mousse | Mousse | Mousse |
|---|---|---|---|---|
| Hair Styling | 6.1 | 6.2 | 6.3 | 6.4 |
| Composition | (wt %) | (wt %) | (wt %) | (wt %) |
| Deionized water | 86.530 | 86.530 | 85.962 | 85.962 |
| Sorbitol | 0.010 | 0.010 | 0.010 | 0.010 |
| PVP | 0.595 | 0.595 | 0.595 | 0.595 |
| Cationic cellulose | 0.709 | 0.709 | 0.709 | 0.709 |
| derivative | | | | |
| (Polyquaternium-4) | | | | |
| LUPAMIN ® 1595 | 1.324 | 1.324 | 0.945 | 0.945 |
| LUPAMIN ® 9095 | | | | |
| LUPAMIN ® 9050 | 3.782 | 2.836 | | 0.945 |
| LUPAMIN ® 9030 | | 0.945 | 1.891 | 0.945 |
| LUPAMIN ® 9010 | | | 2.836 | 2.836 |
| GLYCERINE, CP/USP | 0.010 | 0.010 | 0.010 | 0.010 |
| COCAMIDOPROPYL | 0.020 | 0.020 | 0.020 | 0.020 |
| BETAINE | | | | |
| PPG-12-PEG-50 | 0.176 | 0.176 | 0.176 | 0.176 |
| LANOLIN | | | | |
| PROPYLENE GLYCOL, | 0.002 | 0.002 | 0.002 | 0.002 |
| USP | | | | |
| CYCLOMETHICONE | 0.027 | 0.027 | 0.027 | 0.027 |
| POLYSORBATE 20, | 0.678 | 0.678 | 0.678 | 0.678 |
| WATER | | | | |
| FRAGRANCE | 0.339 | 0.339 | 0.339 | 0.339 |
| ISOBUTANE (AND) | 5.800 | 5.800 | 5.800 | 5.800 |
| PROPANE (Propellant) | | | | |

### Example 7

This example demonstrates the high humidity curl retention properties of exemplary compositions.

Curl retention were measured using a methodology as described in Example 5 above.

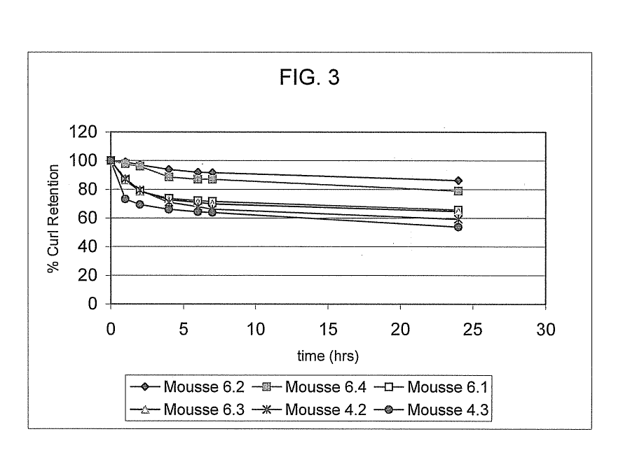

FIG. 3 depicts the percent curl retention over time for Mousse 4.2, Mousse 4.3, Mousse 6.1, Mousse 6.2, Mousse 6.3 and Mousse 6.4. All example compositions achieved at least about 60% curl retention or better after about 2 hours under conditions of about 90% relative humidity at about 75° F.

### Example 8

This example illustrates a process for preparing exemplary mousse compositions. A process as described above in Example 4 was followed.

The ingredients and amounts used (in wt % relative to the total weight of the composition) are summarized in Table 5. The amounts of LUPAMIN® represent the amount of solution as supplied by BASF.

TABLE 5

| Hair Styling | | Mousse 8.1 | Mousse 8.2 | Mousse 8.3 |
|---|---|---|---|---|
| Composition | Phase | (wt %) | (wt %) | (wt %) |
| Deionized water | A | 40.41 | 40.41 | 40.41 |
| Cationic cellulose | A | 0.75 | 0.75 | 0.75 |
| derivative | | | | |
| (Polyquaternium-4) | | | | |
| PVP/DMAEMethac- | A | 3.01 | 3.01 | 3.01 |
| rylate Copolymer | | | | |
| Deionized water | B | 37.79 | 37.79 | 37.79 |
| PVP (20 wt %) | B | 4.01 | 4.01 | 4.01 |
| Disodium EDTA | B | 0.02 | 0.02 | 0.02 |
| LUPAMIN ® 5095 | B | | | 1.00 |
| LUPAMIN ® 9050 | B | | 1.00 | |
| LUPAMIN ® 9030 | B | 2.00 | 2.00 | 2.00 |
| LUPAMIN ® 9010 | B | 4.01 | 3.01 | 3.01 |
| Propylene Glycol | B | 1.00 | 1.00 | 1.00 |
| Sorbitol (70 wt %) | B | 0.50 | 0.50 | 0.50 |
| PPG-5-Ceteth-20 | C | 0.10 | 0.10 | 0.10 |
| Oleth-20 | C | 0.40 | 0.40 | 0.40 |
| Fragrance | C | 0.10 | 0.10 | 0.10 |
| Dimethicone Polyol | C | 0.10 | 0.10 | 0.10 |
| ISOBUTANE (AND) | | 5.80 | 5.80 | 5.80 |
| PROPANE (Propellant) | | | | |

### Example 9

This example demonstrates curl retention under high humidity conditions of exemplary compositions using a methodology as described in Example 5

Curl retentions were measured using the methodology described in Example 5 above.

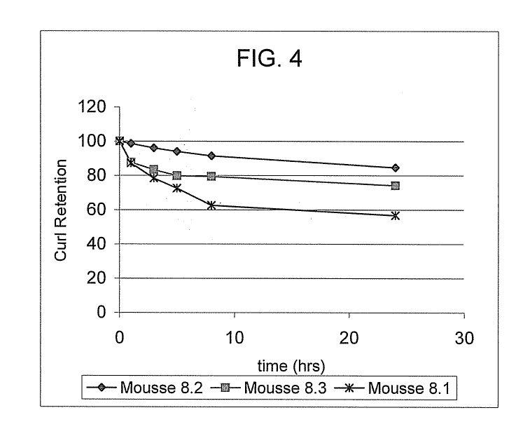

FIG. 4 depicts the percent curl retention over time for Mousse 8.1, Mousse 8.2 and Mousse 8.3. All example compositions achieved at least about 60% curl retention after about 10 hours under conditions of about 90% relative humidity at about 75° F.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A hair-styling composition comprising:
a poly(vinylamine-vinylformamide) copolymer
a polyvinylpyrrolidone, and
an aqueous carrier,
wherein the composition exhibits at least about 50% curl retention after about 2 hours under conditions of about 90% relative humidity at about 75° F.

2. The hair-styling composition of claim 1, wherein the poly(vinylamine-vinylformamide) copolymer comprises a high molecular weight poly(vinylamine-vinylformamide) copolymer and a low molecular weight poly(vinylamine-vinylformamide) copolymer.

3. The hair-styling composition of claim 2, wherein the high molecular weight poly(vinylamine-vinylformamide) copolymer comprises at most about 95 mol % vinylamine monomers.

4. The hair-styling composition of claim 2, wherein the high molecular weight poly(vinylamine-vinylformamide) copolymer comprises at most about 50 mol % vinylamine monomers.

5. The hair-styling composition of claim 2, wherein the high molecular weight poly(vinylamine-vinylformamide) copolymer comprises at most about 30 mol % vinylamine monomers.

6. The hair-styling composition of claim 2, wherein the high molecular weight poly(vinylamine-vinylformamide) copolymer comprises at most about 10 mol % vinylamine monomers.

7. The hair-styling composition of claim 2, wherein the low molecular weight poly(vinylamine-vinylformamide) copolymer comprises about 95 mol % vinylamine monomers.

8. The hair-styling composition of claim 1, further comprising a film former.

9. The hair-styling composition of claim 8, wherein the film former is a quaternized cellulose derivative, a vinylpyrrolidone copolymer, or a combination thereof.

10. The hair-styling composition of claim 1, further comprising a conditioning agent.

11. The hair-styling composition of claim 1, further comprising a surfactant.

12. The hair-styling composition of claim 1, further comprising a propellant.

13. The hair-styling composition of claim 1, wherein the composition is in the form of a mousse or a gel.

14. The hair-styling composition of claim 1, wherein the composition exhibits at least about 60% curl retention after about 2 hours under conditions of about 90% relative humidity at about 75° F.

15. The hair-styling composition of claim 1, wherein the composition exhibits at least about 60% curl retention after about 10 hours under conditions of about 90% relative humidity at about 75° F.

16. The hair-styling composition of claim 1, wherein the composition exhibits at least about 80% curl retention after about 2 hours under conditions of about 90% relative humidity at about 75° F.

17. The hair-styling composition of claim 1, wherein the composition exhibits at least about 80% curl retention after about 10 hours under conditions of about 90% relative humidity at about 75° F.

18. A method of styling mammalian hair, the method comprising contacting the hair with a styling-effective amount of the composition of claim 1, and styling the hair.

19. A method of controlling fizz in mammalian hair, the method comprising contacting the hair with a frizz-controlling effective amount of the composition of claim 1.

20. A personal care product comprising a container, a valve associated with the container, and the composition of claim 1 contained within the container, wherein the valve is adapted to dispense the composition therefrom.
